(19) United States
(12) Patent Application Publication
(10) Pub. No.: US 2025/0163476 A1
(43) Pub. Date: May 22, 2025

(54) RESOURCE RECYCLING METHOD AND RESOURCE RECYCLING MANAGEMENT METHOD

(71) Applicant: KUBOTA CORPORATION, Osaka-shi, Osaka (JP)

(72) Inventors: Futoshi MIZUTANI, Amagasaki-shi (JP); Tsunehisa TANAKA, Amagasaki-shi (JP); Tetsuhiro HASEGAWA, Amagasaki-shi (JP)

(73) Assignee: KUBOTA CORPORATION, Osaka-shi, Osaka (JP)

(21) Appl. No.: 18/838,285

(22) PCT Filed: Feb. 22, 2023
(86) PCT No.: PCT/JP2023/006468

§ 371 Date: Aug. 14, 2024

(30) Foreign Application Priority Data
Mar. 9, 2022 (JP) 2022-035822

(51) Int. Cl. C12P 5/02; C05F 5/00; C05F 17/00

(52) U.S. Cl. CPC C12P 5/023; C05F 5/002; C05F 17/00

(57) ABSTRACT

A resource recycling method that can efficiently utilize a large amount of harvest residues produced in agricultural fields as resources wherein the method includes: a harvest residue collection step of collecting harvest residues produced in an agricultural field; an anaerobic treatment step of causing the harvest residues collected in the harvest residue collection step to undergo methane fermentation; and a returning step of returning fermentation residues produced in the anaerobic treatment step to the agricultural field. The harvest residues collected in the agricultural field are caused to undergo methane fermentation, and the produced methane gas is recovered and effectively utilized as energy, and the fermentation residues are returned to the agricultural field as a compost or fertilizer.

### TECHNICAL FIELD

The present invention relates to a resource recycling method and a resource recycling management method.

### BACKGROUND ART

A large amount of rice straw produced from rice harvest operations has conventionally been plowed back into the soil of an agricultural field as an organic fertilizer or incinerated at the agricultural field. However, methane produced from the decomposition of the plowed-back rice straw in the soil in an anaerobic atmosphere is a greenhouse gas that has a large impact on global warming. Also, the soot and dust and the smoke produced from the incineration of the rice straw at the agricultural field causes air pollution and odor problems. As such, there is a challenge in how to process the large amount of rice straw. Such a challenge is not exclusive to rice straw, but is common to agricultural wastes (referred to herein as “harvest residues”) produced after the harvest of grains harvested in agricultural fields, such as wheat straw.

Patent Literature 1 proposes a methane fermentation method of causing straw crushed into pieces of 10 mm to 100 mm to undergo methane fermentation in a fermentation liquid to recover biogas and also recovering the straw after the fermentation from the digested solution to utilize it as a bedding material.

### CITATION LIST

### Patent Literature

Patent Literature 1: Japanese Patent No. 5567718

### SUMMARY OF INVENTION

### Technical Problem

The methane fermentation method disclosed in Patent Literature 1 is a very desirable technique in terms of effective utilization of biomass resources, but there is room for further innovations in terms of efficiently processing a large amount of harvest residues produced in agricultural fields, including rice straw and wheat straw.

It is an object of the present invention to provide a resource recycling method and a resource recycling management method that can efficiently utilize a large amount of harvest residues produced in agricultural fields as resources.

### Solution to Problem

To achieve the above object, a resource recycling method is provided in accordance with the present invention. As a first feature of the resource recycling method, the method comprises: a harvest residue collection step of collecting harvest residues produced in an agricultural field; an anaerobic treatment step of causing the harvest residues collected in the harvest residue collection step to undergo methane fermentation; and a returning step of returning fermentation residues produced in the anaerobic treatment step to the agricultural field.

The harvest residues collected in agricultural fields are caused to undergo methane fermentation, and the produced methane gas is recovered and effectively utilized as energy, and the fermentation residues are returned to the agricultural fields as a compost or fertilizer. This recycling and utilization of resources can significantly reduce the farming cost.

In addition to the first feature above, the resource recycling method has a second feature that the method further comprises, between the harvest residue collection step and the anaerobic treatment step, a raw material storage step of storing the harvest residues, and the method supplies at least a portion of the harvest residues stored in the raw material storage step to the anaerobic treatment step.

A large amount of harvest residues is collected from agricultural fields due to overlapping harvest periods. Such a large amount of harvest residues can be temporarily stored in the raw material storage step, and if necessary, a portion of the harvest residues can be supplied to the anaerobic treatment step. This allows for, for example, effective utilization of the harvest residues even outside the harvest periods.

In addition to the second feature above, the resource recycling method has a third feature that the raw material storage step is a step of storing the harvest residues collected in the harvest residue collection step dispersedly in a plurality of storage locations.

Even when a large amount of harvest residues is collected at one time, storing the harvest residues dispersedly in multiple storage locations can eliminate the need for securing a large site, which would be required when storing the harvest residues in a single storage location, and can also reduce transportation costs by storing the residues in each nearest storage location.

In addition to the third feature above, the resource recycling method has a fourth feature that the raw material storage step is a step of storing the harvest residues collected in the harvest residue collection step under different storage conditions corresponding to the storage locations.

By varying the storage conditions for the harvest residues according to the storage locations when storing the harvest residues dispersedly in the multiple storage locations, even a large amount of harvest residues can be processed flexibly to meet anticipated future resource demands. Assumable storage conditions include, for example, the cut length of the harvest residues and the amount of storage. For example, when the anaerobic treatment needs to be done early, the harvest residues with a shorter cut length can be used to shorten the hydraulic retention time (HRT), which can consequently increase the amount to be treated of the anaerobic treatment. Adjusting the amount of storage and cut length of the harvest residues in this manner before storing them in different storage locations can accommodate changes in when and how much the anaerobic treatment is to be done.

In addition to the fourth feature above, the resource recycling method has a fifth feature that the method further comprises a management step of managing storage information including the storage locations and the storage conditions to store the harvest residues in the raw material storage step, and managing, based on the storage information, a timing and/or amount of supply of at least a portion of the harvest residues to the anaerobic treatment step.

Storing the harvest residues in different storage locations under different storage conditions depending on the timing when the anaerobic treatment will be required and the amount to be treated required at that time facilitates the management of the timing and amount to be treated of the subsequent anaerobic treatment.

In addition to any one of the first to fifth features above, the resource recycling method has a sixth feature that the method further comprises, between the anaerobic treatment step and the returning step, a fermentation residue storage step of storing the fermentation residues produced in the anaerobic treatment step, and the method returns at least a portion of the fermentation residues stored in the fermentation residue storage step to the agricultural field.

Storing the fermentation residues produced in the anaerobic treatment step allows them to be returned as a compost or fertilizer to the agricultural field in need, when needed.

A resource recycling management method is also provided in accordance with the present invention. As a first feature of the resource recycling management method, the method comprises: a harvest residue collection step of collecting harvest residues produced in an agricultural field; an anaerobic treatment step of causing the harvest residues collected in the harvest residue collection step to undergo methane fermentation; a returning step of returning at least a portion of fermentation residues produced in the anaerobic treatment step to the agricultural field; and a resource recycling management step of adjusting an amount to be treated in the anaerobic treatment step based on an amount and timing of the harvest residues obtained in the harvest residue collection step and a return amount required in the returning step.

In addition to the first feature above, the resource recycling management method has a second feature that the method further comprises a raw material storage step of storing the harvest residues, and the resource recycling management step adjusts storage locations and storage conditions for raw materials in the raw material storage step based on the amount and timing of the harvest residues obtained in the harvest residue collection step and the return amount required in the returning step.

### Advantageous Effects of Invention

As described above, the present invention can provide a resource recycling method and a resource recycling management method that can efficiently utilize a large amount of harvest residues produced in agricultural fields as resources.

### BRIEF DESCRIPTION OF DRAWINGS

FIG. 1 illustrates a method for processing harvest residues produced in agricultural fields.

FIG. 2 illustrates a resource recycling method for harvest residues produced in agricultural fields.

FIG. 3 is a characteristic diagram illustrating correlation between a length L of the harvest residues (rice straw), a hydraulic retention time (HRT), and an amount to be treated.

### DESCRIPTION OF EMBODIMENTS

An anaerobic treatment method, a resource recycling method incorporating the anaerobic treatment method, and a resource recycling management method of the present invention are described below, by way of example with respect to rice straw, which is rice harvest residues produced in agricultural fields.

### Resource Recycling System

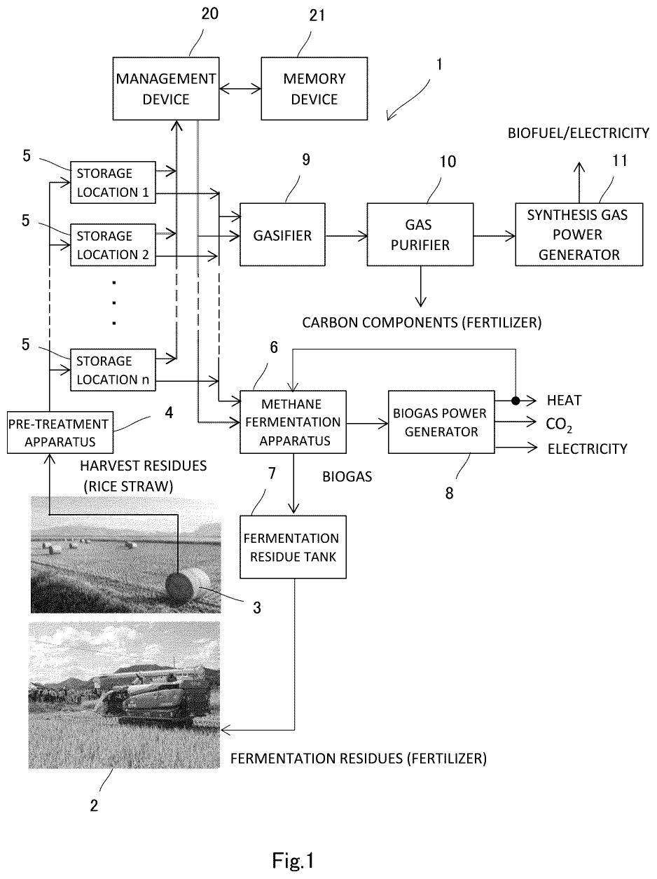

FIG. 1 illustrates a resource recycling system 1 implementing the resource recycling method of the present invention.

The resource recycling system 1 is constructed for each community farm or for multiple community farms in the neighborhood. The resource recycling system 1 includes multiple agricultural fields 2 where rice straw, or harvest residues 3, is produced, multiple storage locations 5 for storing the harvest residues 3, a methane fermentation apparatus 6 for conducting methane fermentation treatment mainly for the harvest residues 3, and a gasifier 9 for generating a synthesis gas from the harvest residues 3. FIG. 1 shows a single methane fermentation apparatus 6 and a single gasifier 9; however, in practice, multiple lines of methane fermentation apparatuses 6 and/or multiple lines of gasifiers 9 may be dispersed or be consolidated in one location, depending on the size of the harvest residues 3 to be received.

The resource recycling system 1 also includes a biogas power generator 8 for generating electricity using methane gas (biogas) generated in the methane fermentation apparatus 6 as a fuel. The electricity generated by the biogas power generator 8 is consumed as electrical energy for the relevant area, and combustion waste heat produced in the biogas power generator 8 is used as a heat source for the methane fermentation apparatus 6 and for greenhouses. Carbon dioxide produced in the biogas power generator 8 is supplied to greenhouses as a raw material gas for photosynthesis.

The methane fermentation apparatus 6 includes, among others, a methane fermentation tank containing a fermentation liquid, a feeder for feeding the harvest residues 3 into the methane fermentation tank, a mixing mechanism for mixing the harvest residues 3 and the fermentation liquid, and a heating mechanism for adjusting the fermentation temperature. A portion of the combustion waste heat produced during power generation is supplied to the heating mechanism to heat the methane fermentation tank to about 55° C. suitable for a thermophilic methane fermentation method, and organic substances are digested under anaerobic conditions to generate the biogas such as methane gas and carbon dioxide.

A fermentation residue tank 7 is provided near the methane fermentation apparatus 6 to store fermentation residues produced in the methane fermentation apparatus 6, and the fermentation residues stored in the fermentation residue tank 7 are returned to the agricultural fields 2 as a compost or fertilizer.

The gasifier 9 includes a reaction tower into which the cut harvest residues 3 are fed. The harvest residues 3 are fluidized and mixed with high-temperature steam and oxygen gas inside the reaction tower to cause a water gas reaction and a water gas shift reaction, thereby generating a synthesis gas containing hydrogen and carbon monoxide. Biochar consisting of silica-containing carbon components is also generated as ash discharged with the synthesis gas.

The water gas reaction refers to an endothermic reaction in which carbon monoxide CO and hydrogen H₂ are generated from solid carbon C contained in the harvest residues 3 and steam H₂O in a high temperature environment at or above 500° C., as shown in the following formula:

C+H₂O→CO+H₂

The water gas shift reaction refers to an exothermic reaction in which carbon dioxide CO₂ and hydrogen H₂ are generated from carbon monoxide CO and steam H₂O in a high temperature environment at or above 800° C., as shown in the following formula:

CO+H₂O→CO₂+H₂

The synthesis gas generated in the gasifier 9 is purified by a gas purifier 10, and the biochar containing carbon components removed from the synthesis gas is returned to the agricultural fields 2 as a compost or fertilizer together with the fermentation residues described above.

The resource recycling system 1 includes a synthesis gas power generator 11 that uses the synthesis gas generated in the gasifier 9 as a fuel to generate electricity. The generated electricity is supplied as electrical energy for the relevant area, and the combustion waste heat produced in the synthesis gas power generator 11 is used as a heat source for the methane fermentation apparatus 6 and for greenhouses. Carbon dioxide produced in the synthesis gas power generator 11 is supplied to greenhouses as a raw material for photosynthesis.

Instead of the synthesis gas power generator 11, the resource recycling system 1 may include an FT synthesis apparatus that uses a synthesis gas consisting of carbon monoxide and hydrogen as a raw material to synthesize liquid hydrocarbon as a fuel through a catalytic reaction. The FT synthesis, which is an abbreviation for the Fischer-Tropsch synthesis, refers to a series of synthesis reaction processes for synthesizing liquid hydrocarbon from carbon monoxide and hydrogen through a catalytic reaction.

At the time of rice harvest, a large amount of rice straw remaining in the agricultural fields 2 after the harvest, or the harvest residues 3, is, for example, packed into a cylindrical shape and collected by a roll baler or the like. Accumulating such a large amount of harvest residues in one location and conducting methane fermentation treatment on it at one time is physically difficult, including the cost of equipment, so even if a large amount of biogas is temporarily obtained, it may not be effectively utilized. Accordingly, separate biogas storage facilities are required.

Thus, multiple storage locations 5 are dispersed throughout the farming areas constituting the community farm. The harvest residues 3 collected in each agricultural field 2 are, if necessary, cut by a chipper shredder or any other crusher that is used as a pre-treatment apparatus 4 and capable of cutting the residues into a predetermined size, before being accumulated in a nearest storage location 5. The above phrase “if necessary” means that such cutting is performed to correspond to the hydraulic retention time (HRT) during methane fermentation, which is adjusted according to various demand forecasts by a management device 20 (described below).

Various forms of storages are provided in the storage location 5, including a roofed storage and an unroofed storage where the residues are stored open-air. The appropriate number of methane fermentation apparatuses 6 and/or gasifiers 9 are provided according to the layout and number of storage locations 5.

A management device 20, which is implemented by a cloud-based server equipped with a memory device 21, is provided to manage the harvest residues 3 for each community farm or for multiple community farms in the neighborhood. In addition to the storage locations and types of storage, the management device 20 manages information such as the start of storage (rice harvest time), cut lengths, varieties, and growers. The resource recycling system 1 is configured such that a manager of the methane fermentation apparatus 6 and a manager of the gasifier 9 can identify the management condition of the harvest residues 3 via respective terminals that can communicate with the management device 20.

The resource recycling system 1 is configured such that the management device 20 creates a storage plan and a utilization plan for the harvest residues 3 based on annual demand forecasts until the following year's harvest, and based on the plans, the harvest residues 3 are stored dispersedly in the multiple storage locations 5 in different crushed conditions and utilized. Uncrushed harvest residues 3 are stacked in cylindrical packages, and harvest residues 3 crushed into a predetermined size are stored in containers such as flexible container bags.

The resource recycling system 1 is configured such that the utilization plan created by the management device 20 and information about the dispersed storage of the harvest residues 3 in the storage locations 5 based on the utilization plan are reported to operators including the managers via respective terminals, and the operators including the managers can properly process the harvest residues 3 based on the utilization plan.

The demand forecasts include a forecast of the amount of electricity required to be generated by the biogas power generator 8 or the synthesis gas power generator 11 for each predetermined period, a forecast of the amount of heat required for a predetermined period when the combustion waste heat produced from the combustor in the biogas power generator 8 or the synthesis gas power generator 11 is used, and a forecast of the amount of fermentation residues required for a predetermined period when the fermentation residues from the methane fermentation apparatus 6 are used as a compost or fertilizer.

For example, when the combustion waste heat produced from the combustor in the biogas power generator 8 or the synthesis gas power generator 11 is used as a heat source for greenhouses, forecasts of the amount and timing of heat required are stored in the memory device 21. Also, for example, when the fermentation residues are returned to the agricultural fields as a fertilizer based on the amount of fertilizer components in the fermentation residues that is analyzed in advance, forecasts of when the fertilizer is to be applied as a base fertilizer, the amount of fertilizer components required, and the amount of fermentation residues returned, as well as forecasts of when the fertilizer is to be applied as an additional fertilizer, the amount of fertilizer components required, and the amount of fermentation residues returned are stored in the memory device 21. In the case of rice cultivation, it is necessary to apply the base fertilizer mainly during soil puddling conducted in April and May and to apply the additional fertilizer mainly around July. The just enough amount of fertilizer required to be applied at such times is stored as a demand forecast in the memory device 21.

### Resource Recycling Method

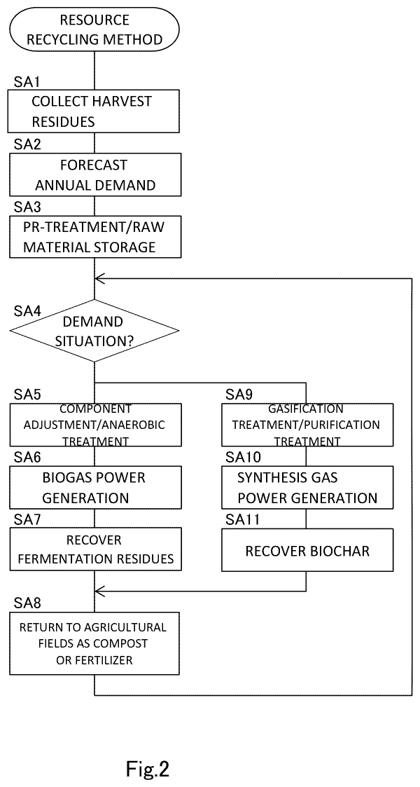

As shown in FIG. 2, the resource recycling method operated by the resource recycling system 1 is configured to execute, among others: a harvest residue collection step (SA1) of collecting the harvest residues 3 produced in the agricultural fields 2; a demand forecasting step (SA2) of forecasting the annual demand for recycled resources using the harvest residues; a raw material storage step (SA3) of storing the harvest residues 3 dispersedly in multiple storage locations 5 based on the forecast demand; an anaerobic treatment step (SA5) of supplying a portion of the stored harvest residues 3 to the methane fermentation apparatus 6 to cause them to undergo methane fermentation based on the demand situation (SA4); and a returning step (SA8) of returning the fermentation residues produced in the anaerobic treatment step to the agricultural fields 2 as a compost or fertilizer.

The harvest residues 3 are caused to undergo methane fermentation in the anaerobic treatment step (SA5), and the produced methane gas is recovered and used as a fuel for biogas power generation (SA6), thus effectively utilizing the energy recovered from the harvest residues 3. The fermentation residues are also recovered (SA7) and returned to the agricultural fields 2 as a compost or fertilizer (SA8). This recycling and utilization of resources can reduce the farming cost.

When a large amount of harvest residues 3 is collected from the agricultural fields 2 due to overlapping harvest periods, the harvest residues 3 can be temporarily stored in the storages in the raw material storage step SA3, and if necessary (SA4), a portion of the harvest residues 3 can be supplied to the anaerobic treatment step SA5. This allows for, for example, effective utilization of the harvest residues 3 even outside the harvest periods. The harvest residues 3 collected from the agricultural fields 2 may be supplied directly to the anaerobic treatment step SA5 without going through storages provided in the storage locations 5.

Preferably, the raw material storage step SA3 is configured to store the harvest residues 3 collected in the harvest residue collection step SA1 under different storage conditions corresponding to the storage locations 5. This is because by varying the storage conditions for the harvest residues 3 according to the storage locations 5 when storing the harvest residues 3 dispersedly in the multiple storage locations 5, even a large amount of harvest residues 3 can be processed flexibly to meet anticipated future resource demands.

Assumable storage conditions include, for example, the cut length of the harvest residues 3 and the amount of storage. For example, when the anaerobic treatment needs to be done early, harvest residues with a shorter cut length can be used to shorten the HRT, which can consequently increase the amount to be treated of the anaerobic treatment. Adjusting the amount of storage and cut length of the harvest residues in this manner as a preprocess before storing them in different storage locations can accommodate changes in when and how much the anaerobic treatment is to be done. When the harvest residues 3 are stored over a long period of time, they can be stored in a long state without being cut, which, taking advantage of the storage period, allows them to property mature under anaerobic conditions to facilitate methane fermentation.

As shown in step SA4, if the management device 20 determines, based on the forecast demand stored in the memory device 21 or an actual demand, that a large amount of additional fertilizer will be needed in the near future, such as one month ahead, the harvest residues may be managed such that those in the storage location 5 where the harvest residues with a shorter cut length are accumulated are supplied to the nearest methane fermentation apparatus 6, and it may be operated with a shorter HRT setting. The amount to be treated of the anaerobic treatment can be thus adjusted, making it possible to obtain fermentation residues with high dissolved content suitable for additional fertilizers in a short period of time.

If the management device 20 determines, based on the forecast demand stored in the memory device 21 or an actual demand, that a large amount of base fertilizer will be needed in a more distant future, such as several months ahead, the harvest residues may be managed such that those in the storage location 5 where the harvest residues 3 with a longer cut length are accumulated are supplied to the nearest methane fermentation apparatus 6 in advance, starting from a time when there is enough time to ensure a sufficient HRT. Thus, the required amount of fermentation residues with high organic content suitable for base fertilizers can be secured until the time when they are needed.

Since the harvest residues 3 contain organic substances such as lignin, which is difficult to decompose in anaerobic condition, and fertilizer ingredients such as silica components, returning the harvest residues 3 to agricultural fields as a compost or fertilizer can effectively restore the soil fertility. The harvest residues 3 have a high C/N ratio and lack elements necessary for methane fermentation, which may make stable methane fermentation impossible. In addition, the harvest residues 3 have small nitrogen and phosphorus components, so that there is an imbalance in nutrients necessary for a compost or fertilizer. Accordingly, the anaerobic treatment in step SA5 preferably involves feeding deficient components, such as copper, iron, nickel, and cobalt, needed by methane fermentation bacteria, as well as nitrogen and phosphorus components recovered from livestock manure and the like to the methane fermentation apparatus 6 to cause methane fermentation, thereby adjusting the components of the harvest residues 3 to make them a well-balanced compost or fertilizer.

As described above, the resource recycling method is configured to execute a management step of managing storage information including the storage locations 5 and the storage conditions to store the harvest residues 3 in the raw material storage step SA3, and managing, based on the storage information, the timing and/or amount of supply of at least a portion of the harvest residues to the anaerobic treatment step SA5.

The management step is executed by the above-described management device 20, which informs the managers and operators in advance to pre-store the harvest residues in different storage locations under different storage conditions depending on the timing when the anaerobic treatment will be required and the amount to be treated required at that time. This facilitates the management of the timing and amount to be treated of the subsequent anaerobic treatment.

The resource recycling method preferably further includes a fermentation residue storage step of storing, in the fermentation residue tank 7, the fermentation residues produced in the anaerobic treatment step SA5 and recovered in the fermentation residue recovery step SA7, and the method is preferably configured to return at least a portion of the fermentation residues stored in the fermentation residue storage step to the agricultural fields 2 in the returning step SA8.

Excess fermentation residues produced in the anaerobic treatment step SA5 are stored in the fermentation residue tank 7, so that they can be returned as a compost or fertilizer to the agricultural field 2 in need, when needed. The fermentation residues with mixed solid and liquid components may be stored in that state, or the fermentation residues may undergo solid-liquid separation to store solid and liquid components separately.

The resource recycling method further includes a gasification treatment step of gasifying the harvest residues 3 collected in the harvest residue collection step SA1 in the gasifier 9 (SA9), and the method is configured to adjust the amount of harvest residues supplied to the gasification treatment step based on the information about demand for fermentation residues determined in step SA4.

A required amount of harvest residues is supplied to the anaerobic treatment step SA5 to yield fermentation residues in an amount that is required in the returning step SA8 based on the information about demand for fermentation residues determined in step SA4, and excess harvest residues 3 are supplied to the gasification treatment step SA9 to generate a synthesis gas. The generated synthesis gas is supplied to the synthesis gas power generator 11 to generate electricity (SA10).

The resource recycling method is preferably configured to adjust the amount of harvest residues supplied to the gasification treatment step SA9 based on the storage information about the harvest residues 3 stored in the raw material storage step SA3.

A large amount of harvest residues 3 collected in the same period is temporarily stored in the raw material storage step SA3, and if necessary, a portion of them is supplied to the anaerobic treatment step SA5 while another portion of them is supplied to the gasification treatment step SA9. This allows for effective utilization of the harvest residues 3 as recycled resources.

For the gasification treatment step SA9, the harvest residues 3 as the raw material need to be cut into small pieces. Accordingly, the management device 20 is configured to manage the harvest residues 3 such that those to be supplied to the gasification treatment step are cut into a predetermined size by the pre-treatment apparatus 4 in step SA3 in advance before they are accumulated in a predetermined storage location 5.

The resource recycling method is configured to recover biochar (SA11), which is a by-product from the gasification treatment step SA9 based on carbon separated in the gas purifier 10, and return it to the agricultural fields 2 together with a compost or fertilizer, which is the fermentation residues (SA8). This allows for efficient recycling and utilization of resources.

### Resource Recycling Management Method

The resource recycling management method according to the present invention is configured to execute: a harvest residue collection step (SA1) of collecting the harvest residues 3 produced in the agricultural fields 2; an anaerobic treatment step (SA5) of causing the harvest residues collected in the harvest residue collection step to undergo methane fermentation; a returning step (SA8) of returning at least a portion of fermentation residues produced in the anaerobic treatment step to the agricultural fields; and a resource recycling management step (SA4) of adjusting the amount to be treated in the anaerobic treatment step based on the amount and timing of the harvest residues 3 obtained in the harvest residue collection step (SA1) and the return amount required in the returning step (SA8).

The resource recycling management method further includes a raw material storage step (SA3) of storing the harvest residues 3. The resource recycling management step (SA4) is configured to adjust the storage locations and storage conditions for the raw material in the raw material storage step (SA3) based on the amount and timing of the harvest residues obtained in the harvest residue collection step (SA1) and the return amount required in the returning step (SA8). The resource recycling management step (SA4) is executed by the above-described management device 20 and memory device 21.

### Anaerobic Treatment Method

The above-described resource recycling method incorporates an anaerobic treatment method of the present invention. That is, the anaerobic treatment method is configured to cause the raw material including the harvest residues 3 produced in the agricultural fields 2 to undergo methane fermentation to utilize fermentation residues produced from the methane fermentation as a compost or fertilizer and to utilize biogas produced from the methane fermentation as an energy source. The anaerobic treatment method is also configured to adjust the cut length of the harvest residues 3 to be supplied to the methane fermentation apparatus 6 based on the information about demand for the compost etc. or the energy source managed by the management device 20.

As already explained above, the time required for methane fermentation is important in terms of matching the demand for methane gas and fermentation residues. Hence, the cut length of the harvest residues 3 is adjusted according to the forecast demand for methane gas and fermentation residues. This enables adjustment of the HTR, which is the time required for methane fermentation, allowing for effective utilization of the organic resources of the harvest residues 3.

Specifically, it is preferable that the cut length or average cut length of the harvest residues 3 to be supplied to the methane fermentation is adjusted to be short in response to an increase in demand for the compost etc. or the energy source.

The methane fermentation process is controlled by operating conditions such as the temperature, hydraulic retention time (HRT), and organic load. By adjusting the cut length or average cut length of the harvest residues to be supplied to the methane fermentation to be short, the HRT can be shortened, which allows for properly responding to an increase in demand for the compost etc. or the energy source.

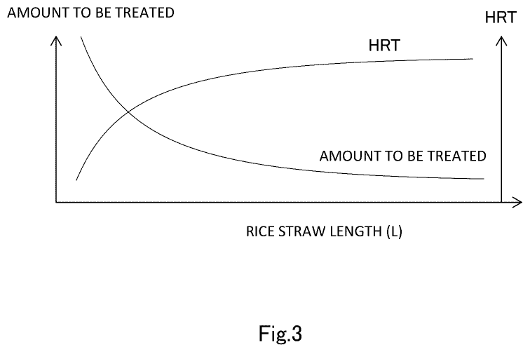

By knowing the relationship between the length of the harvest residues 3 and the HRT required for methane fermentation in advance, the cut length of the harvest residues 3 can be properly adjusted to a value consistent with the target HRT based on this relationship. FIG. 3 shows the relationship between the length L of the rice straw as the harvest residues 3 and the HRT. As a characteristic of the relationship, the shorter the length L of the rice straw, the shorter the HRT, and the HRT gradually saturates as the length L of the rice straw increases. For example, if the length L of the rice straw is a few millimeters, the HRT can be adjusted to about 20 days, and if the length L of the rice straw is a dozen millimeters, the HRT can be adjusted to about 40 days.

The anaerobic treatment method is configured to store the harvest residues 3 separately in multiple cut lengths and, based on the information about the demand for the compost etc. or the energy source, select or load the harvest residues 3 such that those with a predetermined cut length are supplied to the methane fermentation.

By storing the harvest residues 3 separately in multiple cut lengths, those with a predetermined cut length can be selected or loaded from them. This allows the HRT to be adjusted to be consistent with the information about demand for the compost etc. or the energy source.

In the above example, a portion of the harvest residues 3 packed into a cylindrical shape and collected by a roll baler or the like is pulverized by a pulverizer in advance before being stored in the storage location 5. However, they may be crushed or cut into a required size using a crusher when they are subjected to the methane fermentation treatment.

In the above embodiment, a portion of the harvest residues is supplied to the gasifier 9. However, they may be supplied to any other apparatus capable of supplying waste heat and/or electricity and/or returning the products to the agricultural fields, and such apparatuses may include, for example, an apparatus that processes the harvest residues by heating, such as an incinerator, a pyrolysis furnace, and a carbonization furnace.

While the rice straw has been discussed as an example of the harvest residues 3, any agricultural waste produced after the harvest of grains harvested in agricultural fields may be used. As such, the rice straw may contain rice hulls, and wheat straw, cornstalks, etc. may also be used.

It will be appreciated that any of the above-described embodiments is only an example of the present invention. The above descriptions are not limiting the present invention, and the embodiments may be varied as suited, as long as such variations provide the functions and effects of the present invention as well.

### REFERENCE SIGNS LIST

1: Resource recycling system
2: Agricultural field
3: Harvest residue (rice straw)
4: Pre-treatment apparatus
5: Storage location
6: Methane fermentation apparatus
7: Fermentation residue tank
8: Biogas power generator
9: Gasifier
10: Gas purifier
11: Synthesis gas power generator
20: Management device
21: Memory device

1-8. (canceled)

9. A resource recycling method comprising:
a harvest residue collection step of collecting harvest residues produced in an agricultural field;
an anaerobic treatment step of causing the harvest residues collected in the harvest residue collection step to undergo methane fermentation; and
a returning step of returning fermentation residues produced in the anaerobic treatment step to the agricultural field.

10. The resource recycling method according to claim 9, further comprising, between the harvest residue collection step and the anaerobic treatment step, a raw material storage step of storing the harvest residues, wherein
the method supplies at least a portion of the harvest residues stored in the raw material storage step to the anaerobic treatment step.

11. The resource recycling method according to claim 10, wherein the raw material storage step is a step of storing the harvest residues collected in the harvest residue collection step dispersedly in a plurality of storage locations.

12. The resource recycling method according to claim 11, wherein the raw material storage step is a step of storing the harvest residues collected in the harvest residue collection step under different storage conditions corresponding to the storage locations.

13. The resource recycling method according to claim 12, further comprising a management step of managing storage information including the storage locations and the storage conditions to store the harvest residues in the raw material storage step, and managing, based on the storage information, a timing and/or amount of supply of at least a portion of the harvest residues to the anaerobic treatment step.

14. The resource recycling method according to claim 9, further comprising, between the anaerobic treatment step and the returning step, a fermentation residue storage step of storing the fermentation residues produced in the anaerobic treatment step, wherein
the method returns at least a portion of the fermentation residues stored in the fermentation residue storage step to the agricultural field.

15. The resource recycling method according to claim 10, further comprising, between the anaerobic treatment step and the returning step, a fermentation residue storage step of storing the fermentation residues produced in the anaerobic treatment step, wherein
the method returns at least a portion of the fermentation residues stored in the fermentation residue storage step to the agricultural field.

16. The resource recycling method according to claim 11, further comprising, between the anaerobic treatment step and the returning step, a fermentation residue storage step of storing the fermentation residues produced in the anaerobic treatment step, wherein
the method returns at least a portion of the fermentation residues stored in the fermentation residue storage step to the agricultural field.

17. The resource recycling method according to claim 12, further comprising, between the anaerobic treatment step and the returning step, a fermentation residue storage step of storing the fermentation residues produced in the anaerobic treatment step, wherein
the method returns at least a portion of the fermentation residues stored in the fermentation residue storage step to the agricultural field.

18. The resource recycling method according to claim 13, further comprising, between the anaerobic treatment step and the returning step, a fermentation residue storage step of storing the fermentation residues produced in the anaerobic treatment step, wherein
the method returns at least a portion of the fermentation residues stored in the fermentation residue storage step to the agricultural field.

19. A resource recycling management method comprising:
a harvest residue collection step of collecting harvest residues produced in an agricultural field;
an anaerobic treatment step of causing the harvest residues collected in the harvest residue collection step to undergo methane fermentation;
a returning step of returning at least a portion of fermentation residues produced in the anaerobic treatment step to the agricultural field; and
a resource recycling management step of adjusting an amount to be treated in the anaerobic treatment step based on an amount and timing of the harvest residues obtained in the harvest residue collection step and a return amount required in the returning step.

20. The resource recycling management method according to claim 19, further comprising a raw material storage step of storing the harvest residues, wherein
the resource recycling management step adjusts storage locations and storage conditions for raw materials in the raw material storage step based on the amount and timing of the harvest residues obtained in the harvest residue collection step and the return amount required in the returning step.
